(19) United States
(12) Patent Application Publication
(10) Pub. No.: US 2014/0094839 A1
(43) Pub. Date: Apr. 3, 2014

(54) MECHANICAL TENSIONING DEVICE

(71) Applicant: NinePoint Medical, Inc., Cambridge, MA (US)

(72) Inventors: Shekhar Nimkar, Swampscott, MA (US); James W. Moriarty, JR., Georgetown, MA (US)

(73) Assignee: NinePoint Medical, Inc., Cambridge, MA (US)

(21) Appl. No.: 14/036,235

(22) Filed: Sep. 25, 2013

Related U.S. Application Data
(60) Provisional application No. 61/707,424, filed on Sep. 28, 2012.

(51) Int. Cl. A61M 25/10

(52) U.S. Cl. CPC A61M 25/1018
USPC 606/191

(57) ABSTRACT

A mechanical tensioning device includes a housing including a passageway and an opening that is in communication with the passageway. A plunger is movably disposed in the passageway. A first end of the plunger includes an inner member. A connector is coupled to the housing and includes a channel that is in communication with the passageway and a port in communication with the channel. A catheter is coupled to the connector and includes a lumen that is in communication with the channel and an expandable member. The inner member extends through the lumen and into the expandable member such that a distal end of the inner member is fixed to a distal end of the expandable member. The inner member is moveable between a first configuration in which the inner member is curved within the expandable member and a second configuration in which the inner member is less curved within the expandable member.

### TECHNICAL FIELD

The present disclosure generally relates to surgical instruments, and in particular to mechanical tensioning devices configured to center an inner lumen of a balloon catheter.

### BACKGROUND

Balloon catheters are used in a wide variety of medical procedures. Conventional balloon catheters include an inner lumen that extends into a balloon of the balloon catheter. Before the balloon can be properly positioned within a patient's anatomy, or before the balloon catheter can be used in conjunction with another instrument, such as, for example, an endoscope to take images of the patient's anatomy, the inner lumen is typically centered within the balloon. However, centering the inner lumen within the balloon before the balloon is delivered to a selected portion of the patient's anatomy, such as, for example, the patient's esophagus, may cause the inner lumen to bend as the balloon is navigated through non-linear portions of the patient's anatomy and contacts the same. Bending the inner lumen causes the inner lumen of the catheter to become permanently crimped, which typically makes the balloon catheter unusable, for a variety of reasons. Therefore, what is needed is a balloon catheter in which the inner lumen can be selectively curved and straightened within the balloon to allow a medical practitioner to steer the catheter through both linear and non-linear portions of the anatomy of the patient.

### SUMMARY

In one embodiment, in accordance with the principles of the present disclosure, a mechanical tensioning device is provided. The mechanical tensioning device includes a housing comprising an inner surface defining a passageway. A first end of the housing comprises an opening that is in communication with the passageway. The housing comprises a plunger movably disposed in the passageway. A first end of the plunger comprises an inner member extending therefrom. A connector comprises a first end coupled to the first end of the housing. The connector comprises an inner surface defining a channel that is in communication with the passageway. The connector comprises a port in communication with the channel. A catheter comprises a first end coupled to a second end of the connector. The catheter comprises an inner surface defining a lumen that is in communication with the channel. The catheter comprises a second end defining an expandable member. The inner member extends through the opening, the lumen and the expandable member such that a distal end of the inner member is fixed to a distal end the expandable member. The inner member is moveable between a first configuration in which the inner member is curved within the expandable member and a second configuration in which the inner member is less curved within the expandable member.

In one embodiment, in accordance with the principles of the present disclosure, the mechanical tensioning device includes a housing comprising an inner surface defining a passageway. A first end of the housing comprises an opening that is in communication with the passageway. The housing comprises a plunger movably disposed in the passageway. A first end of the plunger comprises an inner member extending therefrom. A second end of the plunger comprises a threaded aperture. A connector comprises a first end coupled to the first end of the housing. The connector comprises an inner surface defining a channel that is in communication with the passageway. The connector comprises a port in communication with the channel. A catheter comprises a first end coupled to a second end of the connector. The catheter comprises an inner surface defining a lumen that is in communication with the channel. The catheter comprises a second end defining an expandable member. The inner member extends through the opening, the lumen and the expandable member such that a distal end of the inner member is fixed to a distal end the expandable member. Moving the plunger within the passageway moves the inner member between a first configuration in which the inner member is curved within the expandable member and a second configuration in which the inner member is less curved within the expandable member. A threaded screw extends through the aperture. A first end of the screw engages a second end of the plunger such that rotating the screw in a first rotational direction causes the plunger to move in a first axial direction and rotating the screw in a second rotational direction opposite the first rotational direction causes the plunger to move in a second axial direction opposite the first axial direction. The screw, the plunger, and the inner member are coaxial.

In one embodiment, in accordance with the principles of the present disclosure, a method for imaging a portion of a patient's anatomy is provided. The method comprises: providing a mechanical tensioning device comprising: a housing comprising an inner surface defining a passageway, a first end of the housing comprising an opening that is in communication with the passageway, the housing comprising a plunger movably disposed in the passageway, a first end of the plunger comprising an inner member extending therefrom, a connector comprising a first end coupled to the first end of the housing, the connector comprising an inner surface defining a channel that is in communication with the passageway, the connector comprising a port in communication with the channel, and a catheter comprising a first end coupled to a second end of the connector, the catheter comprising an inner surface defining a lumen that is in communication with the channel, the catheter comprising a second end defining an expandable member, the inner member extending through the opening, the lumen and the expandable member such that a distal end of the inner member is fixed to a distal end the expandable member; and moving the inner member between a first configuration in which the inner member is curved within the expandable member and a second configuration in which the inner member is less curved within the expandable member.

### BRIEF DESCRIPTION OF THE DRAWINGS

The present disclosure will become more readily apparent from the specific description accompanied by the following drawings, in which:

FIG. 1 is a side view of one embodiment of a mechanical tensioning device in accordance with the principles of the present disclosure;

FIG. 2 is a side, cross-sectional view of the mechanical tensioning device shown in FIG. 1 taken along lines A-A;

FIG. 3 is a side, cross-sectional view of the mechanical tensioning device shown in FIG. 1 taken along lines A-A;

FIG. 4 is an enlarged side, cross-sectional view of the mechanical tensioning device shown in FIG. 1 taken at Detail B;

FIG. 5 is an enlarged side, cross-sectional view of the mechanical tensioning device shown in FIG. 1 taken at Detail C;

FIG. 6 is an enlarged side, cross-sectional view of the mechanical tensioning device shown in FIG. 1 taken at Detail D;

FIG. 7 is a breakaway side, cross-sectional view of the mechanical tensioning device shown in FIG. 1 disposed in a patient;

FIG. 8 is a breakaway side, cross-sectional view of the mechanical tensioning device shown in FIG. 1 disposed in a patient;

FIG. 9 is a side, cross-sectional view of one embodiment of a mechanical tensioning device in accordance with the principles of the present disclosure;

FIG. 10 is a side, cross-sectional view of the mechanical tensioning device shown in FIG. 9;

FIG. 11 is a side, cross-sectional view of one embodiment of a mechanical tensioning device in accordance with the principles of the present disclosure;

FIG. 12 is a side, cross-sectional view of the mechanical tensioning device shown in FIG. 11;

FIG. 13 is a side, cross-sectional view of one embodiment of a mechanical tensioning device in accordance with the principles of the present disclosure; and

FIG. 14 is a side, cross-sectional view of the mechanical tensioning device shown in FIG. 13.

Like reference numerals indicate similar parts throughout the figures.

### DETAILED DESCRIPTION

The exemplary embodiments of a mechanical tensioning device are discussed in terms of a mechanical tensioning device, such as, for example, a balloon catheter that provides mechanical centering of an inner lumen of the balloon catheter. In particular, the inner lumen is connected to a piston, such as, for example, a plunger that is positioned within a housing. The balloon catheter is fixed to a connector that is coupled to the housing. The inner lumen extends through the housing, the connector and the balloon such that a distal end of the inner lumen is fixed to a distal end of the balloon. The plunger is movable within the housing. As the plunger moves, so does the inner lumen.

The inner lumen is movable between a first configuration in which the inner lumen is curved within the balloon and a second configuration in which the inner lumen is less curved (straight or substantially straight) within the balloon. The inner lumen has a first amount of tension when the inner lumen is in the first configuration and the inner lumen has a second amount of tension when the inner lumen is in the second configuration, the first amount of tension being less than the second amount of tension. In some embodiments, the inner lumen is not centered within the balloon when the inner lumen is in the first configuration and the inner lumen is centered or is substantially centered within the balloon when the inner lumen is in the second configuration. That is, the inner lumen extends transverse to an axis defined by the balloon when the inner lumen is in the first configuration and the inner lumen is parallel or is substantially parallel to the axis defined by the balloon when the inner lumen is in the second configuration. In some embodiments, the inner lumen is not parallel to an outer surface of the balloon when the inner lumen is in the first configuration and the inner lumen is parallel to an outer surface of the balloon when the inner lumen is in the second configuration.

The balloon catheter is steered or navigated through the anatomy of a patient to reach a selected linear portion of the patient's anatomy where the balloon catheter is to be positioned, such as, for example, the patient's esophagus. In delivering the balloon cathether from the patient's oral cavity into the patient's esophagus, the medical practitioner will have to steer the balloon through various bends (non-linear portions) of the patient's anatomy. In some embodiments, it is desirable to have the inner lumen in the first configuration while navigating the balloon through the bends. Indeed, because the inner lumen has less tension when in the first configuration, it is more relaxed and hence is better able to bend than when the inner lumen is in the second configuration and has an increased amount of tension. Once the balloon is in the portion of the patient's anatomy selected by the medical practitioner and the portion of the patient's anatomy selected by the medical practitioner is linear or substantially linear, the inner lumen may be moved from the first configuration to the second configuration to straighten the inner lumen within the balloon. The balloon may be moved from a collapsed or unexpanded configuration to an inflated or expanded configuration. In some embodiments, the balloon catheter is placed in line near an entry of an endoscope while light being used to produce images of the patient's anatomy is equidistant from inner walls of the patient's esophagus so as to reduce, if not eliminate, any resulting artifacts that may occur because of unequal scattering of the light.

In some embodiments, the medical practioner may wish to position the balloon in a portion of the patient's anatomy that is curved (non-linear) in connection with the imaging of the patient's esophagus or other portions of the patient's anatomy. In such instances, the medical practitioner may insert the balloon into the curved portion of the patient's anatomy with the inner lumen in the first configuration such that the inner lumen is flexible enough to bend. Because the inner lumen and/or the balloon are flexible, the inner lumen and/or the balloon with assume the shape of the curved portion of the patient's anatomy. That is, at least one of the balloon and the inner lumen have a radius of curvature that is equivalent or substantially equivalent to that of the curved portion of the patient's anatomy. The balloon may then be inflated to move the balloon from a collapsed or unexpanded configuration to an inflated or expanded configuration. In some embodiments, moving the balloon to the inflated or expanded configuration causes an outer surface of the balloon to engage the patient's tissue to fix the balloon relative to the patient's anatomy. In some embodiments, once the balloon is moved to the inflated or expanded configuration, the balloon catheter is placed in line near an entry of an endoscope while light being used to produce images of the patient's anatomy is equidistant from inner walls of the patient's esophagus so as to reduce, if not eliminate any resulting artifacts that may occur because of unequal scattering of the light.

In some embodiments, in addition to adjusting the tension of the inner lumen mechanically, tension may also be provided to the inner lumen by delivering a material into the balloon so as to move the balloon from the collapsed or unexpanded configuration to the inflated or expanded configuration. As the material inflates the balloon, pressure within the balloon increases. As the pressure within the balloon increases, the balloon stretches such that the length of the balloon is greater when the balloon is in the inflated or expanded configuration than when the balloon is in the collapsed or unexpanded configuration. Because the distal end of the inner lumen is fixed to the distal end of the balloon, as the balloon stretches, the inner lumen becomes centered within the balloon. Accordingly, the tension of the inner lumen and hence the degree that the inner lumen is curved and/or centered within the balloon may be adjusted mechanically or by delivering a material into the balloon. In some embodiments, the balloon has a pressure of 5-27 psi when inflated.

In one embodiment, air is inserted into a first opening in the housing to simultaneously inflate the balloon and move the plunger. That is, the insertion of air into the first opening causes the balloon to inflate or expand and the plunger to move within the housing in a first direction such that the inner lumen moves from the first configuration to the second configuration. Air may then be inserted into a second opening opposite the first opening to move the plunger within the housing in a second direction that is opposite the first direction such that the inner lumen moves from the second configuration to the first configuration. In some embodiments, the balloon stretches as the inner lumen moves from the first configuration to the second configuration such that the length of the balloon increases as the inner lumen moves from the first configuration to the second configuration. In some embodiments, the length of the balloon is the same when the inner lumen is in the first configuration or the second configuration.

In one embodiment, the balloon catheter includes a fastener, such as, for example, a screw or thumbscrew inserted into a distal end of the housing. The screw engages a distal end of the piston or plunger such that rotation of the screw in a first direction, such as, for example, clockwise, causes the plunger to move in a first axial direction within the housing. As the plunger moves in the first direction, the inner lumen connected with the plunger also moves such that the inner lumen has a bent or curved configuration within the balloon. In one embodiment, the device includes a biasing member, such as, for example, a spring positioned within a passageway of the housing such that the biasing member engages a proximal end of the plunger and a proximal end of the housing. The biasing member biases the plunger such that the inner lumen is straight or relatively straight (less curved) within the balloon. Rotating the screw in the first direction therefore may require overcoming a force exerted by the biasing member. Rotation of the thumbscrew in a second direction that is opposite the first direction, such as, for example, counterclockwise, causes the screw to back out of the housing and the plunger or piston to move in a second axial direction within the housing that is opposite the first direction. As the plunger moves in the second axial direction, the inner lumen straightens within the balloon (becomes less curved and more centered within the balloon). In some embodiments, as the inner lumen straightens within the balloon, the balloon moves to a stretched configuration. In some embodiments, the inner lumen straightens within the balloon without stretching the balloon. The fastener is offset from an axis defined by the housing.

In one embodiment, the mechanical tensioning device includes a fastener, such as, for example, a screw or mechanical thumbscrew or thumbwheel inserted into a proximal end of a housing of the device. The mechanical thumbscrew or thumbwheel is configured to engage a proximal end of the plunger such that rotation of the mechanical thumbscrew or thumbwheel in one direction, such as, for example, counterclockwise, causes the screw to move within a passageway of the housing and the plunger to move in a first axial direction within the housing. As the plunger moves in the first direction, the inner lumen is curved within the balloon such that the inner lumen has a relaxed configuration. Rotation of the mechanical thumbscrew or thumbwheel in an opposite direction, such as, for example, clockwise, causes the plunger to move in a second direction within the housing, opposite the first direction. As the plunger moves in the second axial direction, the inner lumen straightens (becomes less curved and more centered) within the balloon to provide tension to the inner lumen. In one embodiment, the fastener is coaxial with an axis defined by the housing. In one embodiment, the fastener is offset from an axis defined by the housing.

The mechanical tensioning device of the present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

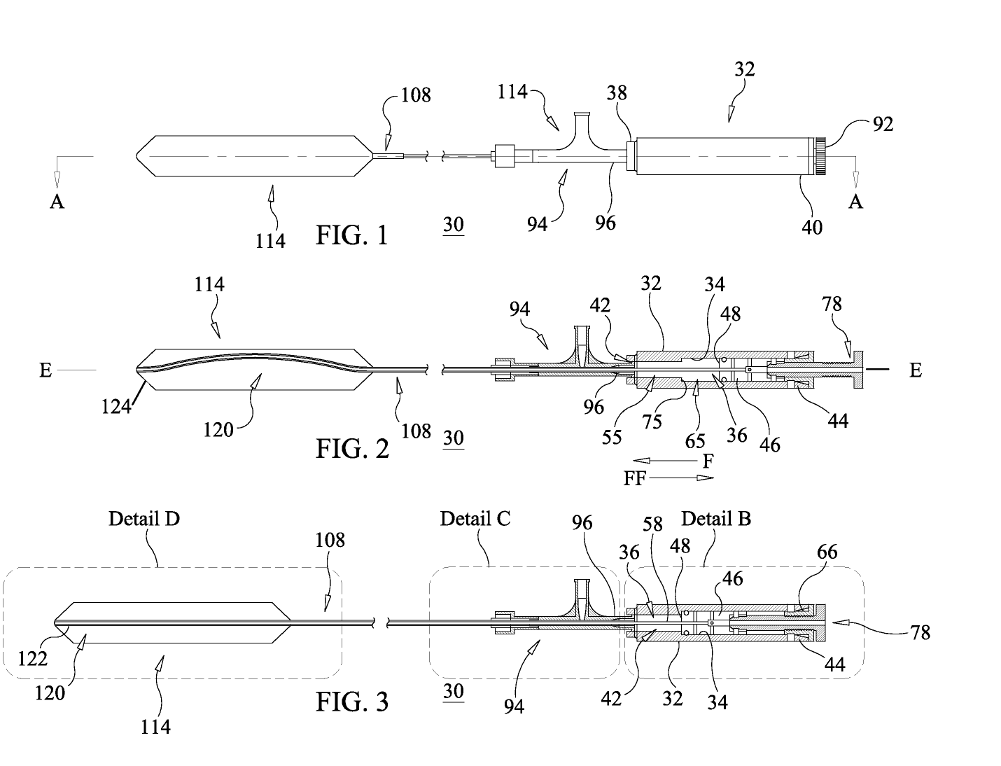

The following discussion includes a description of a mechanical tensioning device. Alternate embodiments are also disclosed. Reference will now be made in detail to the exemplary embodiments of the present disclosure, which are illustrated in the accompanying figures. Turning now to FIGS. 1-8, there are illustrated components of a mechanical tensioning device 30 in accordance with the principles of the present disclosure.

The components of device 30 can be fabricated from biologically acceptable materials suitable for medical applications, including metals, synthetic polymers, ceramics and bone material and/or their composites, depending on the particular application and/or preference of a medical practitioner. For example, the components of device 30, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations. Various components of device 30 may have material composites, including the above materials, to achieve various desired characteristics such as strength, rigidity, elasticity, compliance, biomechanical performance, durability and radiolucency or imaging preference. The components of device 30, individually or collectively, may also be fabricated from a heterogeneous material such as a combination of two or more of the above-described materials. The components of device 30 may be monolithically formed, integrally connected or include fastening elements and/or instruments, as described herein.

Device 30 includes a body, such as, for example, a housing 32. Housing 32 has an inner surface 34 defining a cylindrical passageway 36. Housing 32 extends along a longitudinal axis E between a first end 38 and a second end 40. Housing 32 includes a circular first opening 42 in end 38 and a circular second opening 44 in end 40. Openings 42, 44 are coaxial with one another and axis E and are in communication with passageway 36. Housing 32 has a cylindrical cross sectional configuration. In some embodiments, all or only a portion of housing 32, passageway 36, opening 42 and/or opening 44 may have alternate cross section configurations, such as, for example, circular, oval, oblong, triangular, square, hexagonal, polygonal, irregular, uniform, non-uniform, offset, staggered, and/or tapered.

Housing 32 includes a piston, such as, for example, a cylindrical plunger 46 movably disposed in passageway 36. Plunger 46 is coaxial with axis E and extends along axis E between a first end 48 and a second end 50. Passageway 36 includes a first portion 55 adjacent end 38 having a first maximum width and a second portion 65 adjacent end 40 having a second maximum width that is greater than the first maximum width. An interface between portions 55, 65 defines a flange 75 extending perpendicular to axis E configured to prevent axial translation of plunger 46 in the direction shown by arrow F. That is, end 48 engages flange 75 to prevent axial translation of plunger 46 within passageway 36 in the direction shown by arrow F passed flange 75. In some embodiments, all or only a portion of plunger 46 may have alternate cross section configurations, such as, for example, circular, oval, oblong, triangular, square, hexagonal, polygonal, irregular, uniform, non-uniform, offset, staggered, and/or tapered. In some embodiments, flange 75 may be disposed at alternate orientations relative to axis E, such as, for example, transverse, perpendicular and/or other angular orientations such as acute or obtuse, co-axial and/or may be offset or staggered.

An inner surface 52 of plunger 46 defines a first channel 54 at end 48 and a second channel 56 at end 50. Channels 54, 56 are smooth and free of any recesses and/or projections. Channel 54 includes an inner member 58 disposed therein such that member 58 is fixed to plunger 46. An outer surface 60 of plunger 46 defines a concave circumferential recess 62 having an O-ring 64 disposed therein. O-ring 64 engages surface 34 to create an air tight and/or water tight seal between housing 32 and plunger 46. In some embodiments, member 58 can be variously connected with plunger 46, such as, for example, monolithic, integral connection, frictional engagement, threaded engagement, mutual grooves, screws, adhesive, nails, barbs and/or raised element. In some embodiments, member 58 comprises a flexible material. In some embodiments, member 58 comprises a rigid material. In some embodiments, member 58 comprises a solid configuration that is free of any openings. In some embodiments, member 58 is cannulated to allow a material, such as, for example, a fill material to be delivered through member 58.

Opening 44 includes an insert 66 removably disposed therein. Housing 32 includes a tab 68 that extends through housing 32 and is disposed in a hole 70 that extends through surface 60 to fix insert 66 relative to housing 32 and/or prevent rotation of insert 66 relative to housing 32. An inner surface 72 of insert 66 includes a thread form 74 that engages a thread form 76 of a fastener 78, as will be described. In some embodiments, opening 42 is tapered so as to facilitate insertion of insert 66 into opening 42. In some embodiments, an outer surface 68 of insert 66 forms a friction fit with surface 34. In some embodiments, insert 66 can be variously connected with opening 42, such as, for example, monolithic, integral connection, frictional engagement, threaded engagement, mutual grooves, screws, adhesive, nails, barbs and/or raised element. In some embodiments, insert 66 comprises a different material than does housing 32.

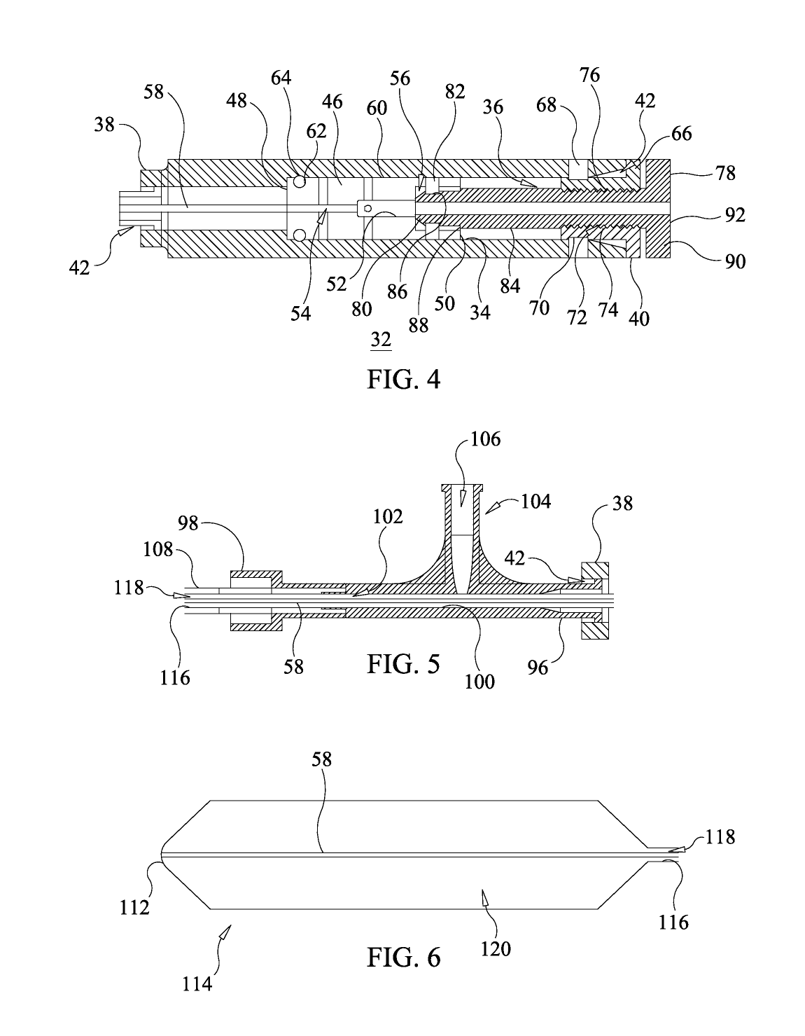

Fastener 78 includes a first end 80 having an outer surface that is free of threads rotatably disposed in channel 56. In some embodiments, plunger 46 includes a retaining member 82 disposed in channel 56 such that member 82 is fixed relative to plunger 46. An inner surface of member 82 engages an outer surface 84 of fastener 78 such that fastener 78 is rotatable relative to member 82, but is prevented from translating relative to plunger 46 in the direction shown by arrow F or the direction shown by arrow FF. In some embodiments, surface 84 includes a circumferential notch 86 adjacent end 80 configured for rotatable disposal of member 82. That is, member 82 is rotatably disposed in notch 86 such that rotating fastener 78 within notch 86 without rotating fastener 78 within hole 70 will not cause plunger 46 to move axially within passageway 36 in the direction shown by arrow F and/or the direction shown by arrow FF. In some embodiments, fastener 78 includes a lip 88 extending perpendicular to axis E that engages an end surface of end 50 when member 82 is disposed in notch 86, as best shown in FIG. 4, to prevent axial translation of fastener 78 relative to plunger 46 in the direction shown by arrow F passed lip 88. In some embodiments, member 82 is a circlip or snap ring. In some embodiments, lip 88 may be disposed at alternate orientations relative to axis E, such as, for example, transverse, perpendicular and/or other angular orientations such as acute or obtuse, co-axial and/or may be offset or staggered.

A second end 90 of fastener 78 includes a thumbwheel 92 that is positioned outside of passageway 36 such that thumbwheel 92 is accessible by a medical practitioner to rotate fastener 78 in a first rotational direction, such as, for example, clockwise, or a second rotational direction, such as, for example, counterclockwise. In some embodiments, thumbwheel 92 includes a plurality of spaced apart ridges that each extend parallel to axis E configured to facilitate gripping of thumbwheel 92 by a medical practitioner. Rotating thumbwheel 92 in a first rotational direction, such as, for example, clockwise, causes fastener 78 to move relative to housing 32 in a first axial direction, as shown by arrow F. As fastener 78 moves relative to housing 32 in the first axial direction, plunger 46 and member 58 also move relative to housing 32 in the first axial direction, as shown in FIG. 3. Rotating thumbwheel 92 in a second rotational direction, such as, for example, counterclockwise, causes fastener 78 to move relative to housing 32 in a second axial direction, as shown by arrow FF. As fastener 78 moves relative to housing 32 in the second axial direction, plunger 46 and member 58 also move relative to housing 32 in the second axial direction, as shown in FIG. 2.

A T-shaped connector 94 includes a first end 96 positioned in opening 42 such that an outer surface of connector 94 engages surface 34 to form a friction fit to couple connector 94 with housing 32. Connector 94 is coaxial with axis E and extends along axis E between end 96 and a second end 98. An inner surface 100 of connector 94 defines a cylindrical channel 102 that is coaxial with axis E. Member 58 extends through channel 102. Connector 94 includes a port 104 extending perpendicular to axis E and defining a cylindrical conduit 106 that is in communication with channel 102. In some embodiments, connector 94 can be variously connected with housing 32, such as, for example, monolithic, integral connection, frictional engagement, threaded engagement, mutual grooves, screws, adhesive, nails, barbs and/or raised element. In some embodiments, port 104 may be disposed at alternate orientations relative to axis E, such as, for example, transverse, perpendicular and/or other angular orientations such as acute or obtuse, co-axial and/or may be offset or staggered. In some embodiments, all or only a portion of channel 102 and/or conduit 106 may have alternate cross section configurations, such as, for example, circular, oval, oblong, triangular, square, hexagonal, polygonal, irregular, uniform, non-uniform, offset, staggered, and/or tapered. In some embodiments, connector 94 is a Tuohy-Borst adapter.

Device 30 includes a catheter 108 extending between a first end 110 and a second end 112 including an expandable member 114, such as, for example a balloon. In some embodiments, balloon 114 is made from a resilient biocompatible material. In one embodiment, balloon 114 comprises a bioresorbable material. In one embodiment, balloon 114 is a compliant balloon comprising polyolefin copolymer (POC). In one embodiment, balloon 114 is a non-compliant balloon comprising polyethylene terapthelate (PET). In one embodiment, catheter 108 is an optical coherence tomography (OCT) catheter. In one embodiment, catheter 108 is an esophageal catheter. An inner surface 116 of catheter 108 defines a lumen 118. Member 58 extends through lumen 118 and into an interior chamber 120 of balloon 114 such that a distal end 122 of member 58 is fixed to a distal end 124 of balloon 114. In some embodiments, end 122 is bonded to end 124. In some embodiments, end 122 is bonded to end 124 using a heat seal.

Catheter 108 is configured to deliver a material, such as, for example, air through a space between an outer surface of member 58 and surface 116 and into chamber 120 and/or provide a pathway for removal of the material from chamber 120. In one embodiment, the material used to fill balloon 114 is air, nitrogen, saline, water and/or combinations thereof. In one embodiment, the material used to fill balloon 114 is a liquid that is configured to cure at or about body temperature. In some embodiments, the material is delivered through conduit 106 and into channel 102. The material moves through channel 102 and the space between the outer surface of member 58 and surface 116 in the direction shown by arrow F until the material is deposited within chamber 120 in a manner that moves balloon 114 from an uninflated, collapsed or deflated configuration to an inflated or expanded configuration.

Member 58 is moveable between a first configuration in which member 58 is relaxed within chamber 120 and has a curved profile, as shown in FIG. 2 and a second configuration in which member 58 is tensioned within chamber 120 and has a less curved or straight profile to provide tension to balloon 114, as shown in FIG. 3. In particular, rotating fastener 78 in a first direction, such as, for example, clockwise, causes end 80 to engage end 50 such that plunger 46 translates within passageway 36 in the direction shown by arrow F. In that member 58 is fixed to plunger 46, as plunger 46 moves in the direction shown by arrow F, so does member 58, such that member 58 moves from the first configuration, shown in FIG. 2, to the second configuration, shown in FIG. 3. That is, moving member 58 from the first orientation to the second orientation removes at least some slack in member 58 such that member 58 is tensioned and becomes straight or substantially straight (less curved). Member 58 hence has a greater amount of tension in the second configuration than in the first configuration. When member 58 is in the first configuration, member 58 is not centered within chamber 120. That is, the portion of member 58 within balloon 114 does not extend parallel to an outer surface of balloon 114. When member 58 is in the second configuration, member 58 is centered or substantially centered within chamber 120. That is, the portion of member 58 within balloon 114 extends parallel or substantially parallel to the outer surface of balloon 114. In some embodiments, balloon 114 comprises a non-compliant material that stretches, at least to some degree, when member 58 is in the second configuration. Because end 122 is fixed to end 124, as balloon 114 stretches, the amount of tension on member 58 increases. In some embodiments, balloon 114 comprises a compliant material that resists stretching when member 58 is in the second configuration such that the length of the balloon is the same when member 58 is in both the first and second configurations.

To move member 58 from the second configuration, shown in FIG. 3 to the first configuration, shown in FIG. 2, fastener 78 is rotated in a second direction that is opposite the first direction, such as, for example, counterclockwise. As fastener 78 is rotated in the second direction, plunger 46 translates within passageway 36 in the direction shown by arrow FF. In that member 58 is fixed to plunger 46, as plunger 46 moves in the direction shown by arrow FF, so does member 58, such that member 58 moves from the second configuration, shown in FIG. 3, to the first configuration, shown in FIG. 2.

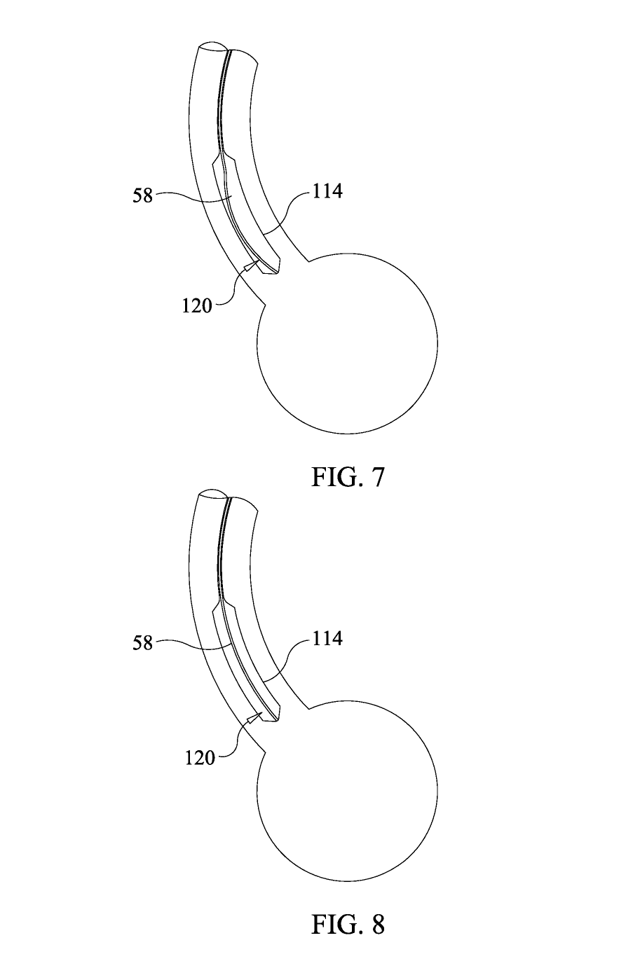

In operation and use, device 30 is inserted into an internal cavity of a patient, such as, for example, the esophagus of the patient, with member 58 in the first configuration, shown in FIG. 2. Device 30 is then navigated to a linear portion of the patient's esophagus to be illuminated and/or imaged using an endoscope, as shown in FIG. 7. It should be appreciated that navigating device 30 within the patient's esophagus while member 58 is in the first configuration provides catheter 108 with flexibility that allows catheter 108 to bend as device 30 is being navigated through bends or turns in the patient's anatomy to reach the linear portion of the patient's esophagus. That is, having member 58 relaxed or less tense positions the portion of member 58 positioned within chamber 120 transverse to an outer surface of balloon 114 and allows catheter 108 to steer through the bends or turns in the esophagus in a manner that prevents device 30 from being damaged.

Once device 30 is in the linear portion of the patient's esophagus, fastener 78 is rotated in a first direction, such as, for example, clockwise to translate plunger 46 within passageway 36 in the direction shown by arrow F such that member 58 moves to the second configuration, shown in FIG. 3. This provides tension to member 58 such that member 58 becomes less curved or straight within chamber 120, as shown in FIG. 8, such that the portion of member 58 within chamber 120 extends parallel to an outer surface of balloon 114. Generally, the more fastener 78 is rotated in the first direction, the greater the tension applied to member 58 is. Accordingly, fastener 78 may be rotated in the first direction a few turns if only a small amount of tension to member 58 is required, for example, if it is desired that member 58 be relaxed within chamber 120 to provide flexibility to balloon 114. Fastener 78 may be further rotated in the first direction a few turns to provide more tension to catheter 108. Fastener 78 may be rotated in the first direction until member 58 moves to the second configuration if it is desired that member 58 be centered or substantially centered within balloon 114. The amount of tension provided to member 58 and the degree the portion of member 58 within chamber 120 can be centered within balloon 114 can hence be selected by controlling the amount fastener 78 is rotated in the first direction to translate plunger 46 within passageway 36 in the direction shown by arrow F. That is, when only a small amount of tension is required and it is desired that the portion of member 58 within chamber 120 be curved, fastener 78 may only be rotated a few turns or less to translate plunger 46 within passageway 36 in the direction shown by arrow F a first distance. Likewise, when greater tension is required and it is desired that the portion of member 58 within chamber 120 be less curved, fastener 78 may be rotated a plurality of turns to translated plunger 46 within passageway 36 in the direction shown by arrow F a second distance that is greater than the first distance.

An inflation source, such as, for example, an air line, may be connected to port 114. An inflation material, such as, for example, air, is delivered from the inflation source and into conduit 106. The material moves from conduit 106 and into the space between the outer surface of member 58 and surface 116. The material moves through the space between the outer surface of member 58 and surface 116 in the direction shown by arrow F such that the material moves into chamber 120 to move balloon 114 from the uninflated, collapsed or deflated configuration to the inflated or expanded configuration. In some embodiments, port 114 extends transverse to axis E such that conduit 106 is angled toward end 98 to better direct the material through channel 102 in the direction shown by arrow F.

In some embodiments, when balloon 114 is in the inflated or expanded configuration, an outer surface of balloon 114 engages the patient's tissue, such as, for example, esophageal tissue to fix balloon 114 relative to the patient's anatomy. In some embodiments, a surgical instrument, such as, for example, an endoscope is introduced into the patient's esophagus, with balloon 114 fixed relative to the patient's anatomy. The endoscope may be fixed to device 30 and selectively positioned relative to the patient's anatomy in any manner desired by the medical practitioner. In some embodiments, the cather is positioned so that the catheter is centered within the patient's esophagus to ensure proper imaging by the endoscope. In some embodiments, device 30 is placed in line near an entry of an endoscope while light being used to produce images of the patient's anatomy is equidistant from inner walls of the patient's esophagus so as to reduce, if not eliminate, any resulting artifacts that may occur because of unequal scattering of the light. The endoscope may be used to take images of the patient's esophagus and/or illuminate the patient's esophagus for imaging thereof or in connection with a surgical procedure. In some embodiments, imaging is performed using a probe, which may or may not be part of the endoscope.

Once the desired images of the patient's anatomy and/or objects within the patient's anatomy have been taken and/or the patient's anatomy illuminated, balloon 114 is moved from the expanded orientation to the collapsed orientation by removing the inflation material from chamber 120. As balloon is moved from the expanded orientation to the collapsed orientation, the outer surface of balloon 114 disengages the patient's tissue such that balloon 114 is no longer fixed relative to the patient's anatomy. Member 58 is moved from the second configuration, shown in FIG. 3, to the first configuration, shown in FIG. 2, to reduce the amount of tension on the portion of member 58 that is positioned within chamber 120 to provide balloon 114 with flexibility necessary to steer or navigate device 30 through turns without breaking or damaging device 30. Device 30 is navigated through the patient's anatomy until device is removed therefrom.

In some embodiments, device 30 is delivered to the linear portion of the patient's anatomy with member 58 in the first configuration. Balloon 114 is then moved from the from the uninflated, collapsed or deflated configuration to the inflated or expanded configuration. As balloon 114 moves to the inflated or expanded configuration, the length of balloon 114 increases. Because catheter 108 is fixed to connector 94 and end 122 is fixed to end 124, expanding the length of balloon 114 causes plunger 46 to move relative to housing 32 in the direction shown by arrow F. As plunger 46 moves relative to housing 32 in the direction shown by arrow F, the portion of member 58 within chamber 120 becomes centered within chamber 120 such that member 58 extends parallel or substantially parallel to the outer surface of balloon 114. That is, expanding balloon 114 from the uninflated, collapsed or deflated configuration to the inflated or expanded configuration will cause plunger 46 to move such that member 58 moves from the first configuration to the second configuration without manipulating fastener 78. This allows a medical practitioner to manipulate the positioning of member 58 by adjusting the position of fastener 78 relative to housing 32 or by inflating balloon 214, as discussed above. In some embodiments, member 58 may be moved from the first configuration to the second orientation by adjusting fastener 78 relative to housing 32 and by inflating balloon 214 and member 58 is moved from the second configuration by deflating balloon 214 and adjusting fastener 78 relative to housing 32. An instrument, such as, for example, an endoscope may be attached to device 30 such that the endoscope is centered relative to the patient's anatomy.

In some embodiments, device 30 is navigated to a non-linear portion of the patient's esophagus for positioning of balloon 11 therein. Device 30 is navigated to the non-linear portion of the patient's esophagus in the same manner that device 30 is navigated to the linear portion of the patient's esophagus discussed above. Device 30 is positioned within the non-linear portion of the patient's esophagus with member 58 is in the first configuration to allow at least one of member 58 and balloon 114 to conform to the shape of the non-linear portion of the patient's esophagus. That is, having member 58 relaxed or less tense allows at least one of member 58 and balloon 114 to bend with the non-linear portion of the patient's esophagus in a manner that prevents device 30 from being damaged.

Once device 30 is in the non-linear portion of the patient's esophagus, balloon 114 is moved from the uninflated, collapsed or deflated configuration to the inflated or expanded configuration in the manner discussed above such that the outer surface of balloon 114 engages the patient's tissue, such as, for example, esophageal tissue to fix balloon 114 relative to the patient's anatomy. Device 30 is placed in line near an entry of an endoscope while light being used to produce images of the patient's anatomy is equidistant from inner walls of the patient's esophagus so as to reduce, if not eliminate any resulting artifacts that may occur because of unequal scattering of the light.

Where this application has listed the steps of a method or procedure in a specific order, it may be possible, or even expedient in certain circumstances, to change the order in which some steps are performed, and it is intended that the particular steps of the method or procedure claim set forth herein below not be construed as being order-specific unless such order specificity is expressly stated in the claim.

In one embodiment, a kit is provided that includes one or more housings, such as, for example, housing 32, one or more connectors, such as, for example, connectors 94, one or more catheters, such as, for example, catheter 108. It is envisioned that the housings, connectors and catheters included in the kit may be variously configured and dimensioned with regard to size, shape, thickness, geometry and material such that the kit includes housings, connectors and catheters having different sizes, shapes, thicknesses, geometries and materials. In one embodiment, the kit includes catheters having different lengths. In one embodiment, the kit includes catheters having different types of balloons, such as, for example, compliant balloons and non-compliant balloons. In one embodiment, the kit includes connectors and/or housings having different lengths. In one embodiment, the kit includes a case for the various components of the kits and/or a set of instructions.

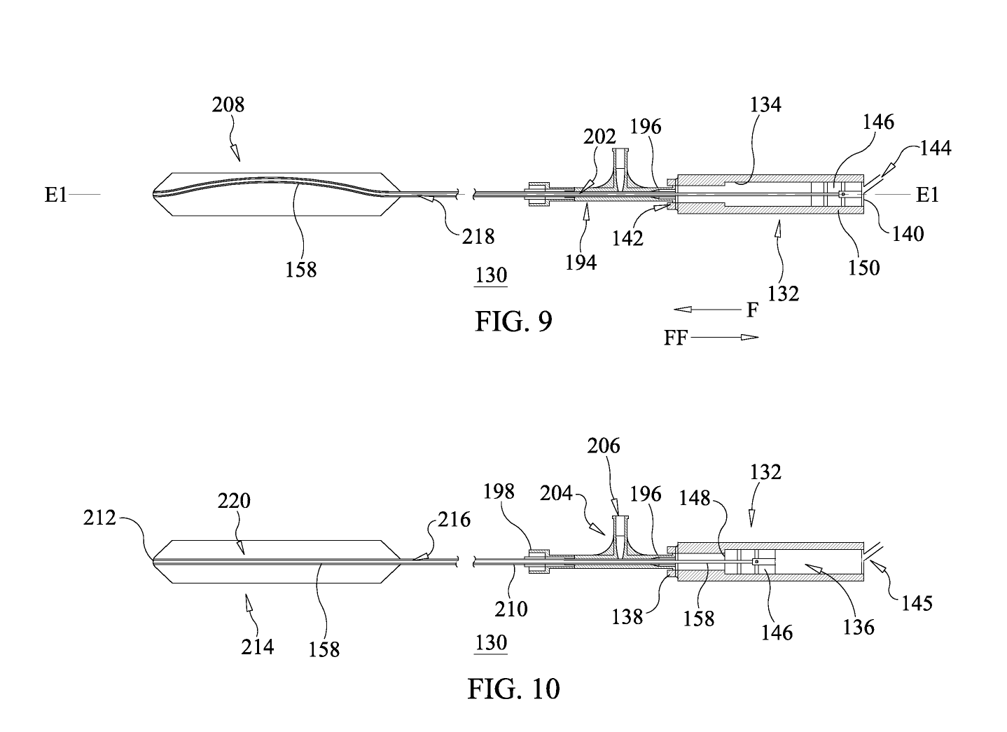

In one embodiment, shown in FIGS. 9 and 10, a device 130 similar to device 30 is provided in accordance with the principles of the present disclosure. Device 130 includes a body, such as, for example, a housing 132 similar to housing 32. Housing 132 has an inner surface 134 defining a cylindrical passageway 136. Housing 132 extends along a longitudinal axis E1 between a first end 138 and a second end 140. Housing 132 includes a first opening 142 in end 138 and a second opening 144 in end 140 that is offset from axis E1. In some embodiments, opening 144 extends through a valve 145. Openings 142, 144 are in communication with passageway 136.

Housing 132 includes a piston, such as, for example, a cylindrical plunger 146 similar to plunger 46 movably disposed in passageway 136. Plunger 146 is coaxial with axis E1 and extends along axis E1 between a first end 148 and a second end 150. In some embodiments, passageway 136 includes a flange similar to flange 75 configured to prevent axial translation of plunger 146 in the direction shown by arrow F. An inner member 158 is disposed within plunger 146 such that member 158 is fixed to plunger 146. An outer surface of plunger 146 defines a concave circumferential recess similar to 62 having an O-ring 164 disposed therein.

A connector 194 includes a first end 196 positioned in opening 142 such that an outer surface of connector 194 engages surface 134 to form a friction fit to couple connector 194 with housing 132. Connector 194 is coaxial with axis E1 and extends along axis E1 between end 196 and a second end 198. An inner surface of connector 194 defines a channel 202 that is coaxial with axis E1. Member 158 extends through channel 202. Connector 194 includes a port 204 extending perpendicular to axis E1 and defining a cylindrical conduit 206 that is in communication with channel 202.

Device 130 includes a catheter 208 similar to catheter 108 extending between a first end 210 and a second end 212 including an expandable member 214, such as, for example a balloon. An inner surface 216 of catheter 208 defines a lumen 218. Member 158 extends through lumen 218 and into an interior chamber 220 of balloon 214 such that a distal end of member 158 is fixed to a distal end of balloon 214. Catheter 208 is configured to deliver a material, such as, for example, air through a space between an outer surface of member 158 and surface 216 and into chamber 220 and/or provide a pathway for removal of the material from chamber 220.

In some embodiments, the material is delivered through conduit 206 and into channel 202. The material moves through channel 202 and the space between the outer surface of member 158 and surface 216 in the direction shown by arrow F until the material is deposited within chamber 220 in a manner that moves balloon 214 from an uninflated, collapsed or deflated configuration to an inflated or expanded configuration. The material simultaneously moves through channel 202 and the space between the outer surface of member 158 and surface 216 in the direction shown by arrow FF to move plunger 146 in the direction shown by arrow FF. That is, delivering the inflation material into port 206 causes plunger 146 to translate in the direction shown by arrow FF such that end 150 is positioned adjacent end 140, as shown in FIG. 9. When end 150 is positioned adjacent end 140, member 158 is curved within chamber 220 such that member 158 is in a first configuration. When member 158 is in the first position, member 158 is relaxed (not tense) within chamber 220 to provide flexibility to balloon 214. Material, such as, for example, air may be delivered into valve 145 through opening 144 to move plunger 146 within passageway 136 in the direction shown by arrow FF until end 150 is spaced apart from end 140 and member 158 extends into chamber 220 a second distance that is greater than the first distance, to move member 158 to a second configuration in which member 158 is less curved within chamber 220 to provide tension to balloon 214, as shown in FIG. 10.

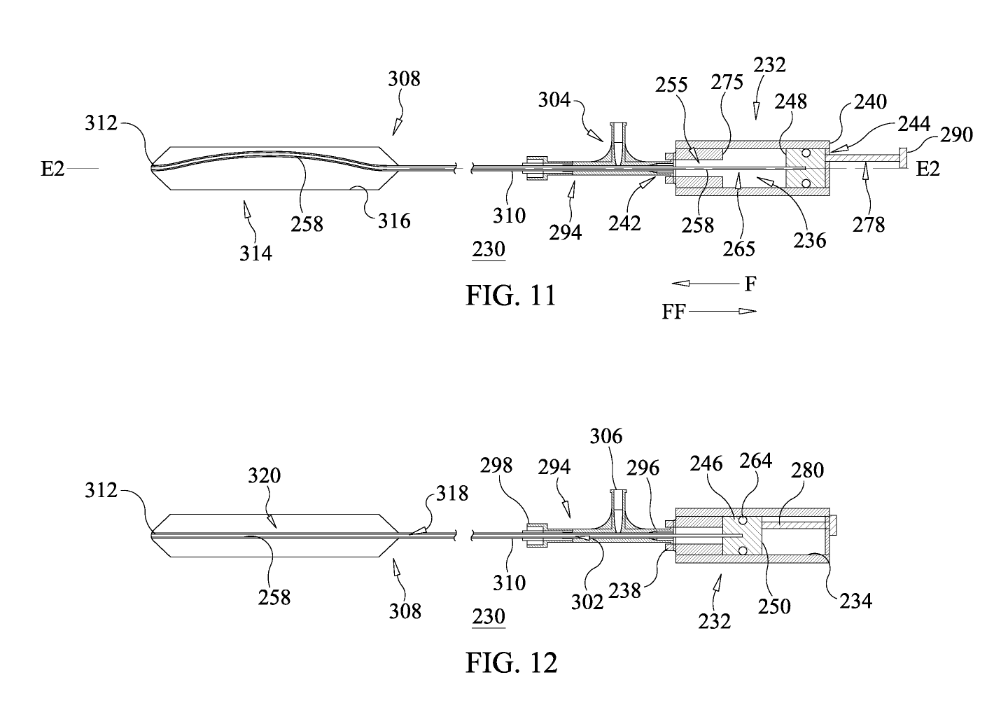

In one embodiment, shown in FIGS. 11 and 12, a device 230 similar to devices 30, 130 is provided in accordance with the principles of the present disclosure. Device 230 includes a body, such as, for example, a housing 232 similar to housings 32, 132. Housing 232 has an inner surface 234 defining a cylindrical passageway 236. Housing 232 extends along a longitudinal axis E2 between a first end 238 and a second end 240. Housing 232 includes a first opening 242 in end 238 and a second opening 244 in end 240. Opening 242 is coaxial with axis E2 and is in communication with passageway 236. Opening 244 is offset from axis E2 and is in communication with passageway 236.

Housing 232 includes a piston, such as, for example, a plunger 246 similar to plungers 46, 146 movably disposed in passageway 236. Plunger 246 is coaxial with axis E2 and extends along axis E2 between a first end 248 and a second end 250. Passageway 236 includes a first portion 255 adjacent end 238 having a first maximum width and a second portion 265 adjacent end 240 having a second maximum width that is greater than the first maximum width. An interface between portions 255, 265 defines a flange 275 similar to flange 75 extending perpendicular to axis E2 configured to prevent axial translation of plunger 246 in the direction shown by arrow F. That is, end 248 engages flange 275 to prevent axial translation of plunger 246 within passageway 236 in the direction shown by arrow F passed flange 275. Plunger 246 includes an inner member 258 that is fixed to plunger 246. In some embodiments, an outer surface of plunger 246 defines a concave circumferential recess similar to recess 62 having an O-ring 264 disposed therein. O-ring 264 engages surface 234 to create an air tight and/or water tight seal between plunger 246 and housing 232. A threaded fastener 278 is inserted into opening 244 such that threads on an outer surface of fastener 278 engage threads on a surface that define opening 244. Fastener 278 includes a first end 280 that engages end 250.

A second end 290 of fastener 278 includes a thumbwheel that is positioned outside of passageway 236 such that the thumbwheel is accessible by a medical practitioner to rotate fastener 278 in a first rotational direction, such as, for example, clockwise, or a second rotational direction, such as, for example, counterclockwise. Rotating fastener 278 in a first rotational direction, such as, for example, clockwise, causes fastener 278 to move relative to housing 232 in a first axial direction, as shown by arrow F. As fastener 278 moves relative to housing 232 in the first axial direction, plunger 246 and member 258 also move relative to housing 232 in the first axial direction. Rotating fastener 278 in a second rotational direction, such as, for example, counterclockwise, causes fastener 278 to move relative to housing 232 in a second axial direction, as shown by arrow FF. As fastener 278 moves relative to housing 232 in the second axial direction, plunger 246 and member 258 also move relative to housing 232 in the second axial direction.

A T-shaped connector 294 includes a first end 296 positioned in opening 242 such that an outer surface of connector 294 engages surface 234 to form a friction fit to couple connector 294 with housing 232. Connector 294 is coaxial with axis E2 and extends along axis E2 between end 296 and a second end 298. An inner surface of connector 294 defines a cylindrical channel 302 that is coaxial with axis E2. Member 258 extends through channel 302. Connector 294 includes a port 304 extending perpendicular to axis E2 and defining a cylindrical conduit 306 that is in communication with channel 302.

Device 230 includes a catheter 308 similar to catheters 108, 208 extending between a first end 310 and a second end 312 including an expandable member 314, such as, for example a balloon. An inner surface 316 of catheter 308 defines a lumen 318. Member 258 extends through lumen 318 and into an interior chamber 320 of balloon 314 such that a distal end of member 358 is fixed to a distal end of balloon 314. Catheter 308 is configured to deliver a material, such as, for example, air through a space between an outer surface of member 258 and surface 316 and into chamber 320 and/or provide a pathway for removal of the material from chamber 320. The material is delivered through conduit 306 and into channel 302. The material moves through channel 302 and the space between the outer surface of member 258 and surface 316 in the direction shown by arrow F until the material is deposited within chamber 320 in a manner that moves balloon 314 from an uninflated, collapsed or deflated configuration to an inflated or expanded configuration.

Member 258 is moveable between a first configuration in which member 258 is curved within chamber 320, as shown in FIG. 11 and a second configuration in which member 258 is less curved within chamber 320 to provide tension to member, as shown in FIG. 12. In particular, to move member 258 from the second configuration, shown in FIG. 12, to the first configuration, shown in FIG. 11, fastener 278 is rotated in a first direction, such as, for example, counterclockwise such that fastener 278 backs out of opening 244 and end 290 is spaced apart from end 240, as shown in FIG. 11. A material, such as, for example, air is delivered into conduit 306 such that the material moves through channel 302 in the direction shown by arrow FF until the material exerts a force on end 248 to move plunger 246 within passageway 236 in the direction shown by arrow FF.

To move member 258 from the first configuration, shown in FIG. 11 to the second configuration, shown in FIG. 12, fastener 278 is rotated relative to housing 232 in a second direction, such as, for example, clockwise, such that plunger 246 translates within passageway 236 in the direction shown by arrow F. In that member 258 is fixed to plunger 246, as plunger 246 moves in the direction shown by arrow F, so does member 258. The number of times fastener 278 is rotated in the second direction is directly proportional to the amount plunger 246 translates in the direction shown by arrow F. That is, the more times fastener 278 is rotated in the second direction, the more plunger 246 translates within the direction shown by arrow F. It should be appreciated that plunger 246 may translate within passageway 236 in the direction shown by arrow F by rotating fastener 278 in the second direction until end 248 engages flange 275.

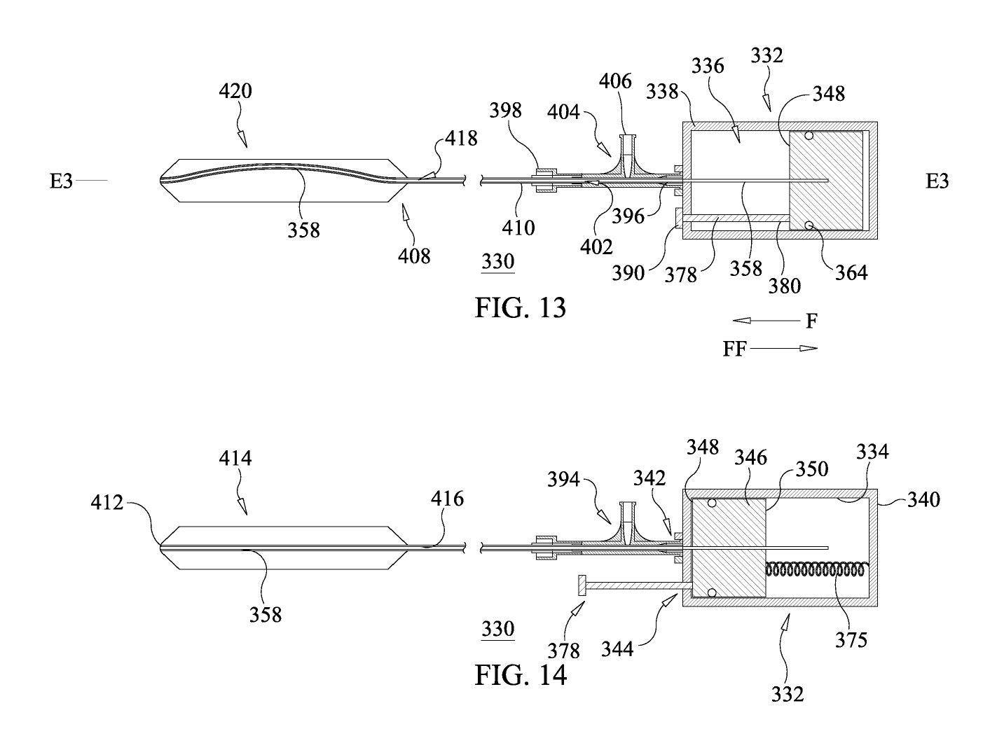

In one embodiment, shown in FIGS. 13 and 14, a device 330 similar to devices 30, 130, 230 is provided in accordance with the principles of the present disclosure. Device 330 includes a body, such as, for example, a housing 332 similar to housings 32, 132, 232. Housing 332 has an inner surface 334 defining a cylindrical passageway 336. Housing 332 extends along a longitudinal axis E3 between a first end 338 and a second end 340. Housing 332 includes a first opening 342 in end 338 and a second opening 344 in end 338 that is spaced apart from opening 342. Opening 342 is coaxial with axis E3 and is in communication with passageway 336. Opening 344 is offset from axis E3 and is in communication with passageway 336.

Housing 332 includes a piston, such as, for example, a plunger 346 similar to plungers 46, 146, 246 movably disposed in passageway 336. Plunger 346 is coaxial with axis E3 and extends along axis E3 between a first end 348 and a second end 350. Plunger 346 includes an inner member 358 that is fixed to plunger 346. In some embodiments, an outer surface of plunger 346 defines a concave circumferential recess similar to recess 62 having an O-ring 364 disposed therein. O-ring 364 engages surface 334 to create an air tight and/or water tight seal between plunger 346 and housing 332. A threaded fastener 378 is inserted into opening 244 such that threads on an outer surface of fastener 378 engage threads on a surface that define opening 344. Fastener 378 includes a first end 380 that engages end 348.

A second end 390 of fastener 378 includes a thumbwheel that is positioned outside of passageway 336 such that the thumbwheel is accessible by a medical practitioner to rotate fastener 378 in a first rotational direction, such as, for example, clockwise, or a second rotational direction, such as, for example, counterclockwise. Rotating fastener 378 in a first rotational direction, such as, for example, clockwise, causes fastener 378 to move relative to housing 332 in a first axial direction, as shown by arrow F. As fastener 378 moves relative to housing 332 in the first axial direction, plunger 346 and member 358 also move relative to housing 332 in the first axial direction. Rotating fastener 378 in a second rotational direction, such as, for example, counterclockwise, causes fastener 378 to move relative to housing 332 in a second axial direction, as shown by arrow FF. As fastener 378 moves relative to housing 332 in the second axial direction, plunger 346 and member 358 also move relative to housing 332 in the second axial direction. In one embodiment, device 330 includes a biasing member, such as, for example, a spring 375 positioned within passageway 336 between end 350 and end 340 configured to bias plunger 346 in the direction shown by arrow FF.

A T-shaped connector 394 includes a first end 396 positioned in opening 342 such that an outer surface of connector 394 engages surface 334 to form a friction fit to couple connector 394 with housing 332. Connector 394 is coaxial with axis E3 and extends along axis E3 between end 396 and a second end 398. An inner surface of connector 394 defines a channel 402 that is coaxial with axis E3. Member 358 extends through channel 402. Connector 394 includes a port 404 extending perpendicular to axis E3 and defining a conduit 406 that is in communication with channel 402.

Device 330 includes a catheter 408 extending between a first end 410 and a second end 412 including an expandable member 414, such as, for example a balloon. An inner surface 416 of catheter 408 defines a lumen 418. Member 358 extends through lumen 418 and into an interior chamber 420 of balloon 414 such that a distal end of member 358 is fixed to a distal end of balloon 214. Catheter 408 is configured to deliver a material, such as, for example, air through a space between an outer surface of member 358 and surface 416 and into chamber 420 and/or provide a pathway for removal of the material from chamber 420. The material is delivered through conduit 406 and into channel 402. The material moves through channel 402 and the space between the outer surface of member 358 and surface 416 in the direction shown by arrow F until the material is deposited within chamber 420 in a manner that moves balloon 414 from an uninflated, collapsed or deflated configuration to an inflated or expanded configuration.

Member 358 is moveable between a first configuration in which member 358 is curved within chamber 420, as shown in FIG. 13 and a second configuration in which member 358 is less curved within chamber 420 to provide tension to member 358, as shown in FIG. 14. Since member 375 is configured to bias plunger 346 in the direction shown by arrow FF, member 358 is biased to the second configuration, as shown in FIG. 14. To move member 358 from the second configuration, shown in FIG. 14, to the first configuration, shown in FIG. 13, fastener 378 is rotated in a first direction, such as, for example, clockwise such that fastener 378 is inserted into opening 344 and end 390 engages end 338 as shown in FIG. 13. To move member 258 from the first configuration, shown in FIG. 13 to the second configuration, shown in FIG. 14, fastener 378 is rotated relative to housing 332 in a second direction, such as, for example, counterclockwise, such that fastener 378 backs out of opening 344. As fastener 378 backs out of opening 344, member 375 exerts a force on end 350 such that plunger 346 translates within passageway 336 in the direction shown by arrow FF. In that member 358 is fixed to plunger 346, as plunger 346 moves in the direction shown by arrow FF, so does member 358. The number of times fastener 378 is rotated in the second direction is directly proportional to the amount plunger 346 translates in the direction shown by arrow FF. That is, the more times fastener 378 is rotated in the second direction, the more plunger 346 translates within the direction shown by arrow FF.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A mechanical tensioning device comprising:
a housing comprising an inner surface defining a passageway, a first end of the housing comprising an opening that is in communication with the passageway, the housing comprising a plunger movably disposed in the passageway, a first end of the plunger comprising an inner member extending therefrom;
a connector comprising a first end coupled to the first end of the housing, the connector comprising an inner surface defining a channel that is in communication with the passageway, the connector comprising a port in communication with the channel; and
a catheter comprising a first end coupled to a second end of the connector, the catheter comprising an inner surface defining a lumen that is in communication with the channel, the catheter comprising a second end defining an expandable member,
wherein the inner member extends through the opening, the lumen and the expandable member such that a distal end of the inner member is fixed to a distal end the expandable member, the inner member being moveable between a first configuration in which the inner member is curved within the expandable member and a second configuration in which the inner member is less curved within the expandable member.

2. A mechanical tensioning device as recited in claim 1, wherein moving the plunger within the passageway moves the inner member between the first and second configurations.

3. A mechanical tensioning device as recited in claim 2, wherein:
the housing comprises a second end comprising a threaded aperture; and
a threaded screw extends through the aperture, a first end of the screw engaging a second end of the plunger such that rotating the screw in a first rotational direction causes the plunger to move in a first axial direction and rotating the screw in a second rotational direction opposite the first rotational direction causes the plunger to move in a second axial direction opposite the first axial direction.

4. A mechanical tensioning device as recited in claim 3, wherein the first end of the screw is rotatably disposed in a cavity in the second end of the plunger such that rotating the screw within the cavity without rotating the screw within the aperture will not cause the plunger to move axially within the passageway.

5. A mechanical tensioning device as recited in claim 3, wherein the aperture is defined by an inner surface of an insert, the insert being removably positioned in the passageway such that an outer surface of the insert engages the inner surface of the housing and the insert is fixed relative to the housing.

6. A mechanical tensioning device as recited in claim 5, further comprising a removable tab extending through the housing and into the outer surface of the insert to fix the insert relative to the housing.

7. A mechanical tensioning device as recited in claim 3, wherein the screw, the plunger, and the inner member are coaxial.

8. A mechanical tensioning device as recited in claim 3, wherein the inner surface of the housing defines a flange having a width that is less than that of the plunger such that the flange engages the first end of the plunger to prevent axial movement of the plunger in one of the first and second axial directions.

9. A mechanical tensioning device as recited in claim 3, wherein:
the housing extends along a longitudinal axis between the first and second ends of the housing; and
the screw is offset from the longitudinal axis.

10. A mechanical tensioning device as recited in claim 2, wherein:
the first end of the housing comprises a threaded aperture; and
a threaded screw extends through the aperture, a first end of the screw engaging the first end of the plunger such that rotating the screw in a first rotational direction causes the plunger to move in a first axial direction and rotating the screw in a second rotational direction opposite the first rotational direction causes the plunger to move in a second axial direction opposite the first axial direction.

11. A mechanical tensioning device as recited in claim 10, wherein:
the housing extends along a longitudinal axis between the first end and an opposite second end; and
the screw is offset from the longitudinal axis.

12. A mechanical tensioning device as recited in claim 2, wherein:
the housing comprises a second end opposite the first end of the housing;
the housing extends along a longitudinal axis between the first and second ends of the housing;
the second end of the housing comprises a valve; and
delivering a material into the port causes the plunger to move in a first axial direction and delivering a material into the valve causes the plunger to move in a second axial direction that is opposite the first axial direction.

13. A mechanical tensioning device as recited in claim 1, wherein:
a space between an outer surface of the inner member and the inner surface of the catheter defines a conduit; and
a material is delivered into the port such that the material moves through the conduit and into the chamber to move the expandable member from a collapsed configuration to an expanded configuration.

14. A mechanical tensioning device as recited in claim 1, wherein:
the expandable member defines an axis;
the inner member extends transverse to the axis when the inner member is in the first configuration; and
the inner member is coaxial with the axis when the inner member is in the second configuration.

15. A mechanical tensioning device as recited in claim 1, wherein the inner member is straight when the inner member is in the second configuration.

16. A mechanical tensioning device comprising:
a housing comprising an inner surface defining a passageway, a first end of the housing comprising an opening that is in communication with the passageway, the housing comprising a plunger movably disposed in the passageway, a first end of the plunger comprising an inner member extending therefrom, a second end of the plunger comprising a threaded aperture;
a connector comprising a first end coupled to the first end of the housing, the connector comprising an inner surface defining a channel that is in communication with the passageway, the connector comprising a port in communication with the channel; and
a catheter comprising a first end coupled to a second end of the connector, the catheter comprising an inner surface defining a lumen that is in communication with the channel, the catheter comprising a second end defining an expandable member,
wherein the inner member extends through the opening, the lumen and the expandable member such that a distal end of the inner member is fixed to a distal end the expandable member, the inner member being moveable between a first configuration in which the inner member is curved within the expandable member and a second configuration in which the inner member is less curved within the expandable member,
wherein a threaded screw extends through the aperture, a first end of the screw engaging a second end of the plunger such that rotating the screw in a first rotational direction causes the plunger to move in a first axial direction and rotating the screw in a second rotational direction opposite the first rotational direction causes the plunger to move in a second axial direction opposite the first axial direction, and
wherein the screw, the plunger, and the inner member are coaxial.

17. A mechanical tensioning device as recited in claim 16, wherein:
the first end of the screw is rotatably disposed in a cavity in the second end of the plunger such that rotating the screw within the cavity without rotating the screw within the aperture will not cause the plunger to move axially within the passageway; and
the expandable member is a balloon.

18. A method for imaging a portion of a patient's anatomy, comprising:
providing a mechanical tensioning device comprising:
a housing comprising an inner surface defining a passageway, a first end of the housing comprising an opening that is in communication with the passageway, the housing comprising a plunger movably disposed in the passageway, a first end of the plunger comprising an inner member extending therefrom,
a connector comprising a first end coupled to the first end of the housing, the connector comprising an inner surface defining a channel that is in communication with the passageway, the connector comprising a port in communication with the channel, and
a catheter comprising a first end coupled to a second end of the connector, the catheter comprising an inner surface defining a lumen that is in communication with the channel, the catheter comprising a second end defining an expandable member, the inner member extending through the opening, the lumen and the expandable member such that a distal end of the inner member is fixed to a distal end the expandable member; and
moving the inner member between a first configuration in which the inner member is curved within the expandable member and a second configuration in which the inner member is less curved within the expandable member.

19. A method as recited in claim 18, wherein moving the inner member comprises moving the plunger within the passageway to move the inner member between the first and second configurations.

20. A method as recited in claim 19, wherein:
the housing comprises a second end comprising a threaded aperture; and
a threaded screw extends through the aperture such that the screw, the plunger, and the inner member are coaxial, a first end of the screw engaging a second end of the plunger such that rotating the screw in a first rotational direction causes the plunger to move in a first axial direction and rotating the screw in a second rotational direction opposite the first rotational direction causes the plunger to move in a second axial direction opposite the first axial direction.
